(19) United States
(12) Patent Application Publication
(10) Pub. No.: US 2002/0133168 A1
(43) Pub. Date: Sep. 19, 2002

(54) Applicator and methods for placing a trabecular shunt for glaucoma treatment

(76) Inventors: Gregory T. Smedley, Irvine, CA (US); Morteza Gharib, San Moreno, CA (US); Hosheng Tu, Newport Coast, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/101,548

(22) Filed: Mar. 18, 2002

Related U.S. Application Data
(60) Provisional application No. 60/276,609, filed on Mar. 16, 2001.

(51) Int. Cl. A61F 2/14

(52) U.S. Cl. 606/108; 604/8; 623/905

(57) ABSTRACT

Disclosed are an apparatus and method for placing a fluid shunt, for the treatment of glaucoma, from inside the anterior chamber of an eye, through the trabecular meshwork, and into Schlemm's canal. The apparatus can include a handpiece having a distal end and a proximal end; an elongate tip connected to the distal end of said handpiece, the elongate tip having a distal portion and being configured to be placed through a corneal incision and into an anterior chamber of said eye; a holder attached to the distal portion of the elongate tip, the holder configured to hold and release said inlet section of the trabecular shunt; and an actuator on the handpiece that actuates the holder to release the inlet section of the trabecular shunt from the holder.

### BACKGROUND OF THE INVENTION

The present invention generally relates to medical devices and methods for reducing intraocular pressure in the animal eye by permitting aqueous humor to flow out of the anterior chamber through a surgically implanted pathway. More particularly, the present invention relates to an applicator and methods for placing a trabecular shunt for glaucoma treatment.

The human eye is a specialized sensory organ capable of light reception and able to receive visual images. The trabecular meshwork serves as a drainage channel and is located in anterior chamber angle formed between the iris and the cornea. The trabecular meshwork maintains a balanced pressure in the anterior chamber of the eye by draining aqueous humor from the anterior chamber.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (“aqueous”) to enter Schlemm's canal, which then empties into aqueous collector channels in the posterior wall of Schlemm's canal and then into aqueous veins, which form the episcleral venous system. Aqueous humor is a transparent liquid that fills the region between the cornea, at the front of the eye, and the lens. The aqueous humor is continuously secreted by the ciliary body around the lens, so there is a constant flow of aqueous humor from the ciliary body to the eye's front chamber. The eye's pressure is determined by a balance between the production of aqueous and its exit through the trabecular meshwork (major route) or uveal scleral outflow (minor route). The trabecular meshwork is located between the outer rim of the iris and the back of the cornea, in the anterior chamber angle. The portion of the trabecular meshwork adjacent to Schlemm's canal (the juxtacanilicular meshwork) causes most of the resistance to aqueous outflow.

Glaucoma is grossly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous humor from the anterior chamber of the eye. Open-angle glaucoma is any glaucoma in which the angle of the anterior chamber remains open, but the exit of aqueous through the trabecular meshwork is diminished. The exact cause for diminished filtration is unknown for most cases of open-angle glaucoma. Primary open-angle glaucoma is the most common of the glaucomas, and it is often asymptomatic in the early to moderately advanced stage. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

Current therapies for glaucoma are directed at decreasing intraocular pressure. Medical therapy includes topical ophthalmic drops or oral medications that reduce the production or increase the outflow of aqueous. However, these drug therapies for glaucoma are sometimes associated with significant side effects, such as headache, blurred vision, allergic reactions, death from cardiopulmonary complications, and potential interactions with other drugs. When drug therapy fails, surgical therapy is used. Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if the morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure).

For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy/Trabeculotomy: Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Trabeculopuncture: Q-switched Neodynium (Nd) YAG lasers also have been investigated as an optically invasive technique for creating fall-thickness holes in trabecular meshwork. However, the relatively small hole created by this trabeculopuncture technique exhibits a filling-in effect and fails.

Goniophotoablation/Laser Trabecular Ablation: Goniophotoablation is disclosed by Berlin in U.S. Pat. No. 4,846,172 and involves the use of an excimer laser to treat glaucoma by ablating the trabecular meshwork. This was demonstrated not to succeed by clinical trial. Hill et al. used an Erbium:YAG laser to create full-thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage: This is an ab interno (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and non-penetrating trabeculectomy (NPT) are two new variations of filtering surgery. These are ab externo (from the outside), major ocular procedures in which Schlemm's canal is surgically exposed by making a large and very deep scleral flap. In the VC procedure, Schlemm's canal is cannula ted and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous humor is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous humor through the surgical opening will continue. The risk of placing a glaucoma drainage device also includes hemorrhage, infection, and diplopia (double vision).

Examples of implantable shunts and surgical methods for maintaining an opening for the release of aqueous humor from the anterior chamber of the eye to the sclera or space beneath the conjunctiva have been disclosed in, for example, U.S. Pat. No. 6,059,772 to Hsia et al., and No. 6,050,970 to Baerveldt.

All of the above surgeries and variations thereof have numerous disadvantages and moderate success rates. They involve substantial trauma to the eye and require great surgical skill in creating a hole through the full thickness of the sclera into the subconjunctival space. The procedures are generally performed in an operating room and have a prolonged recovery time for vision.

The complications of existing filtration surgery have prompted ophthalmic surgeons to find other approaches to lowering intraocular pressure.

The trabecular meshwork and juxtacanilicular tissue together provide the majority of resistance to the outflow of aqueous and, as such, are logical targets for surgical removal in the treatment of open-angle glaucoma. In addition, minimal amounts of tissue are altered and existing physiologic outflow pathways are utilized.

As reported in Arch. Ophthalm. (2000) 118:412, glaucoma remains a leading cause of blindness, and filtration surgery remains an effective, important option in controlling the disease. However, modifying existing filtering surgery techniques in any profound way to increase their effectiveness appears to have reached a dead end. The article further states that the time has come to search for new surgical approaches that may provide better and safer care for patients with glaucoma.

Therefore, there is a great clinical need for a method of treating glaucoma that is faster, safer, and less expensive than currently available modalities.

### SUMMARY OF THE INVENTION

Glaucoma surgical morbidity would greatly decrease if one were to bypass the focal resistance to outflow of aqueous only at the point of resistance, and to utilize remaining, healthy aqueous outflow mechanisms. This is in part because episcleral aqueous humor exerts a backpressure that prevents intraocular pressure from going too low, and one could thereby avoid hypotony. Thus, such a surgery would virtually eliminate the risk of hypotony-related maculopathy and choroidal hemorrhage. Furthermore, visual recovery would be very rapid, and the risk of infection would be very small, reflecting a reduction in incidence from 2-5% to about 0.05%.

Co-pending applications, Ser. No. 09/549,350, filed Apr. 14, 2000, entitled APPARATUS AND METHOD FOR TREATING GLAUCOMA, and Ser. No. 09/704,276, filed Nov. 1, 2000, entitled GLAUCOMA TREATMENT DEVICE, disclose devices and methods of placing a trabecular shunt ab interno, i.e., from inside the anterior chamber through the trabecular meshwork, into Schlemm's canal. Both co-pending patent applications are incorporated herein by reference.

Techniques performed in accordance with aspects herein may be referred to generally as “trabecular bypass surgery.” Advantages of this type of surgery include lowering intraocular pressure in a manner which is simple, effective, disease--site-specific, and can potentially be performed on an outpatient basis.

Generally, trabecular bypass surgery (TBS) creates an opening, a slit, or a hole through trabecular meshwork with minor microsurgery. TBS has the advantage of a much lower risk of choroidal hemorrhage and infection than prior techniques, and it uses existing physiologic outflow mechanisms. In some aspects, this surgery can potentially be performed under topical or local anesthesia on an outpatient basis with rapid visual recovery. To prevent “filling in” of the hole, a biocompatible elongated device is placed within the hole and serves as a stent. U.S. patent application Ser. No. 09/549,350, filed Apr. 14, 2000, the entire contents of which are incorporated herein by reference, discloses trabecular bypass surgery.

### SUMMARY OF THE INVENTION

As described in U.S. patent applications Ser. No. 09/549,350, filed Apr. 14, 2000, and Ser. No. 09/704,276, filed Nov. 1, 2000, a trabecular shunt for transporting aqueous humor is provided. The trabecular shunt includes a hollow, elongate tubular element, having an inlet section and an outlet section. The outlet section may optionally include two segments or elements, adapted to be positioned and stabilized inside Schlemm's canal. In one embodiment, the device appears as a “T” shaped device.

One aspect of the invention includes a delivery apparatus for placing a trabecular shunt through a trabecular meshwork of an eye, the shunt having an inlet section and an outlet section, the delivery apparatus including a handpiece having a distal end and a proximal end; an elongate tip connected to the distal end of the handpiece, the elongate tip having a distal portion and being configured to be placed through a corneal incision and into an anterior chamber of the eye; a holder attached to the distal portion of the elongate tip, the holder configured to hold and release the inlet section of the trabecular shunt; and an actuator on the handpiece that actuates the holder to release the inlet section of the trabecular shunt from the holder.

In some embodiments, the holder comprises a clamp. In some embodiments, the apparatus further comprises a spring within the handpiece that is configured to be loaded when the shunt is being held by the holder, the spring being at least partially unloaded upon actuating the actuator, allowing for release of the shunt from the holder.

In various embodiments, the clamp comprises a plurality of claws configured to exert a clamping force onto the inlet section of the shunt. The holder may also comprise a plurality of flanges.

In some embodiments, the distal portion of the elongate tip is made of a flexible material. This can be a flexible wire. The distal portion can have a deflection range, preferably of about 45 degrees from the long axis of the handpiece.

The delivery apparatus can further comprise an irrigation port in the elongate tip.

Some aspects include a method of placing a trabecular shunt through a trabecular meshwork of an eye, the shunt having an inlet section and an outlet section, including advancing a delivery apparatus holding the trabecular shunt through an anterior chamber of the eye and into the trabecular meshwork, placing part of the shunt through the trabecular meshwork and into a Schlemm's canal of the eye; and releasing the shunt from the delivery apparatus.

In various embodiments, the method includes using a delivery apparatus that comprises a handpiece having a distal end and a proximal end; an elongate tip connected to the distal end of the handpiece, the elongate tip having a distal portion and being configured to be placed through a corneal incision and into an anterior chamber of the eye; a holder attached to the distal portion of the elongate tip, the holder configured to hold and release the inlet section of the trabecular shunt; and an actuator on the handpiece that actuates the holder to release the inlet section of the trabecular shunt from the holder.

In one aspect, the trabecular shunt is removably attached to a delivery apparatus (also known as “applicator”). When the trabecular shunt is deployed from the delivery apparatus into the eye, the outlet section is positioned in substantially opposite directions inside Schlemm's canal. In one embodiment, a deployment mechanism within the delivery apparatus includes a push-pull type plunger. In some embodiments, the delivery applicator may be a guidewire, an expandable basket, an inflatable balloon, or the like.

Among the advantages of trabecular bypass surgery is its simplicity. The microsurgery may potentially be performed on an outpatient basis with rapid visual recovery and greatly decreased morbidity. There is a lower risk of infection and choroidal hemorrhage, and there is a faster recovery, than with previous techniques.

For purposes of summarizing the invention, certain aspects, advantages, and novel features of the invention are described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIGS. 1 and 2 are schematic cross sections of a trabecular shunt and applicator.

FIG. 3 is a schematic cross section of a fluid-pressure or pneumatic release embodiment of the trabecular shunt applicator.

FIG. 4 is a schematic cross section of a trabecular shunt applicator with a hinge-release mechanism.

FIG. 5 is an oblique elevational view of a trabecular shunt applicator with a retractable blade mechanism.

FIG. 6 is an oblique elevational view of a trabecular shunt retrieval device with a claw grasp mechanism.

FIGS. 7A and 7B are schematic cross sections of a trabecular punch device.

FIGS. 8A and 8B are close-up elevational views of the trabecular shunt retrieval device utilizing a claw grasp mechanism.

FIGS. 9A through 9D illustrate an adhesive mechanism for release of the trabecular shunt from the applicator.

FIGS. 10A and 10B are schematic cross sections of a plunger release mechanism for the trabecular shunt applicator.

FIGS. 11A and 11B show a hook-and-eye mechanism for release of the trabecular shunt from its applicator.

FIG. 12A and 12B are elevational views of a magnetic release mechanism for the trabecular shunt applicator.

FIGS. 13A and 13B are schematic cross sections of a screw release mechanism for the trabecular shunt applicator.

FIGS. 14A and 14B are elevational views of a release mechanism for the trabecular shunt applicator utilizing an elastic band.

FIGS. 16A and 16B are schematic cross sectional views of a pin release mechanism for the trabecular shunt applicator.

FIGS. 17A through 17B demonstrate several breakaway mechanisms for the trabecular shunt applicator.

FIG. 18 is a schematic cross section view of a wedge configuration for the trabecular shunt and applicator.

FIG. 9 is a schematic cross section of a spring loaded release mechanism for the trabecular shunt applicator.

FIGS. 20A, 20B and 21 are elevational views of a catch-release mechanism for the trabecular shunt applicator.

FIGS. 22A and 22B demonstrate a suction release mechanism for the trabecular shunt applicator.

FIG. 23 is an oblique elevational view of an articulating arm embodiment of the trabecular shunt retrieval device.

FIGS. 24 and 24B are elevational views of a control arm and trabeculotomy device for the trabecular shunt applicator.

FIGS. 25A through 25C are schematic oblique elevational views of various trabecular meshwork punching and drilling devices.

### DETAILED DESCRIPTION OF THE PREFERRED EMBODIMENT

FIG. 1 illustrates one embodiment of a trabecular shunt applicator 2. The applicator 2 comprises an outer tube 4 and inner tube 6, and two or more flanges 8 at the distal end of the inner tube 6. These flanges 8 can hold the inlet section of trabecular shunt 10 in place while the inner tube 6 is in a retracted position within the outer tube 4 of the applicator 2. When the inner tube 6 is pushed distally (in the direction of the arrows) relative to the outer tube 4, the flanges 8 hold less tightly to the shunt 10, allowing it to be dislodged from the inner tube 6.

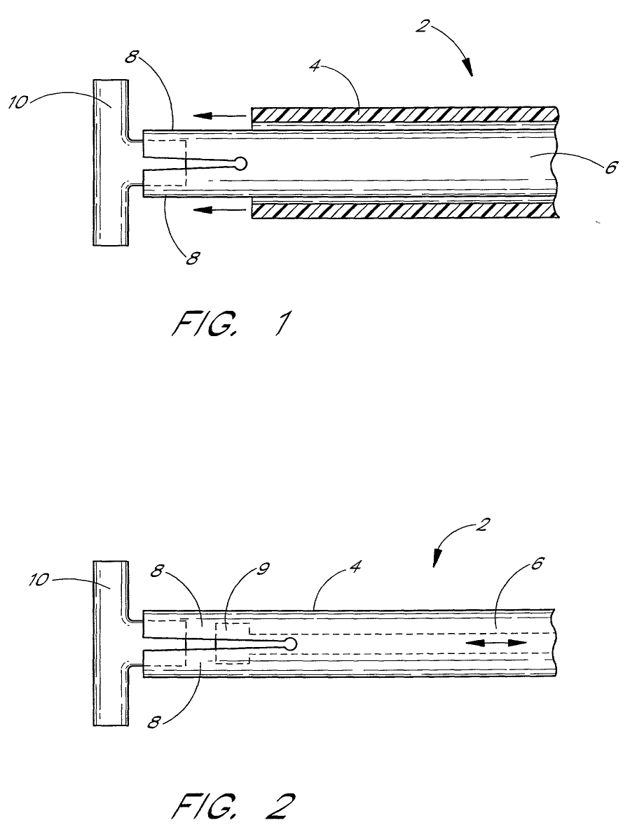

FIG. 2 demonstrates another embodiment of the trabecular shunt applicator 2. In this embodiment, the trabecular shunt 10 is held by the flanges 8 of the inner tube 6. A plunger 9 can move forward and backward (arrows) within the inner tube 6. When the plunger 9 is advanced distally, towards the trabecular shunt 10, the trabecular shunt 10 may be dislodged from the flanges 8 and left in position in the trabecular meshwork of the patient's eye.

Another embodiment of the trabecular shunt applicator is illustrated in FIG. 3. In this embodiment, the shunt 10 is held in place by a pneumatic tube 12. The pneumatically-actuated clamp utilizes a fluid (gas or liquid) to channel the actuation force rather than the mechanical linkage used in some other embodiments. This pneumatic tube 12 comprises an inner wall 16 and an outer wall 14. Between the inner wall 16 and outer wall 14 lies an inner cavity 18. Within the inner cavity 18 fluid can flow (arrows). When fluid flows into the inner cavity 18 under pressure, inner wall 16 and outer wall 14 straighten, causing the distal ends 20 of the pneumatic tube 18 move away (curved arrows) from the shunt 10. Pressurizing the lumen causes the end-effectors (the distal ends 20) to open (Bourdon Tube type of actuator) and releases the shunt 10. In this case, the spring loading is in the closing direction and it is forced open by pneumatic pressure to release the shunt 10. Pressurization could be accomplished by a variety of methods, including pressing a small bladder with a fingertip. When the distal ends 20 of the pneumatic tube 18 do so, they can release the shunt 10 within the patients eye.

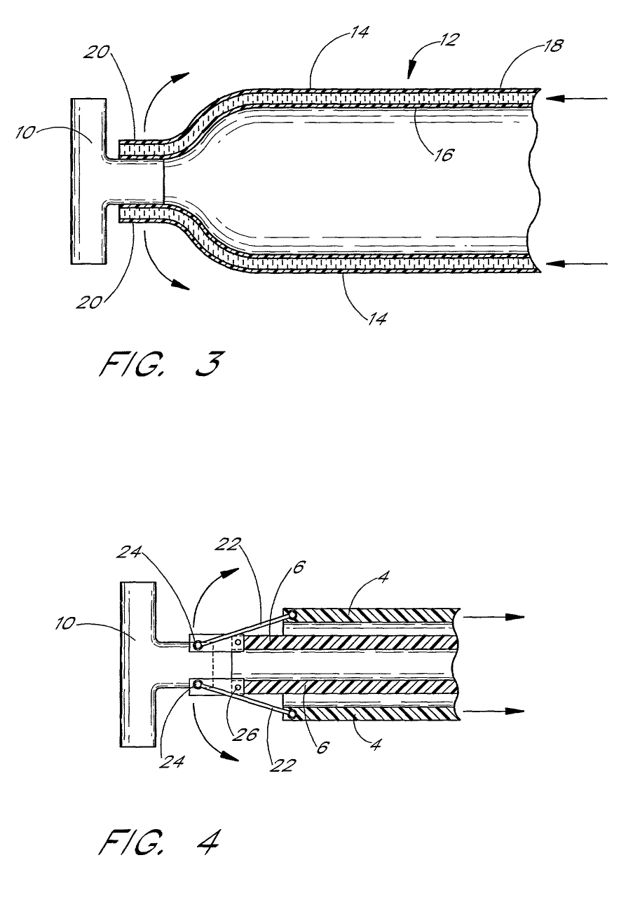

Another embodiment of the trabecular shunt applicator is shown in FIG. 4. In this embodiment two or more holders 24 hold the shunt 10 in place. Rods 22 extend from the outer tube 4 to the holders 24. When the outer tube 4 is retracted proximally relative to the inner tube 6 (straight arrows), the rods 22 exert traction on the holders 24, pulling them outwardly (curved arrows), away from the shunt 10. As the outer tube 4 is retracted further relative to the inner tube 6, the holders 24 release the trabecular shunt 10, leaving the trabecular shunt 10 in place in the eye. The holders 24 may be attached to the inner tube via hinges 26, pivots, or any other acceptable means known to those skilled in the art.

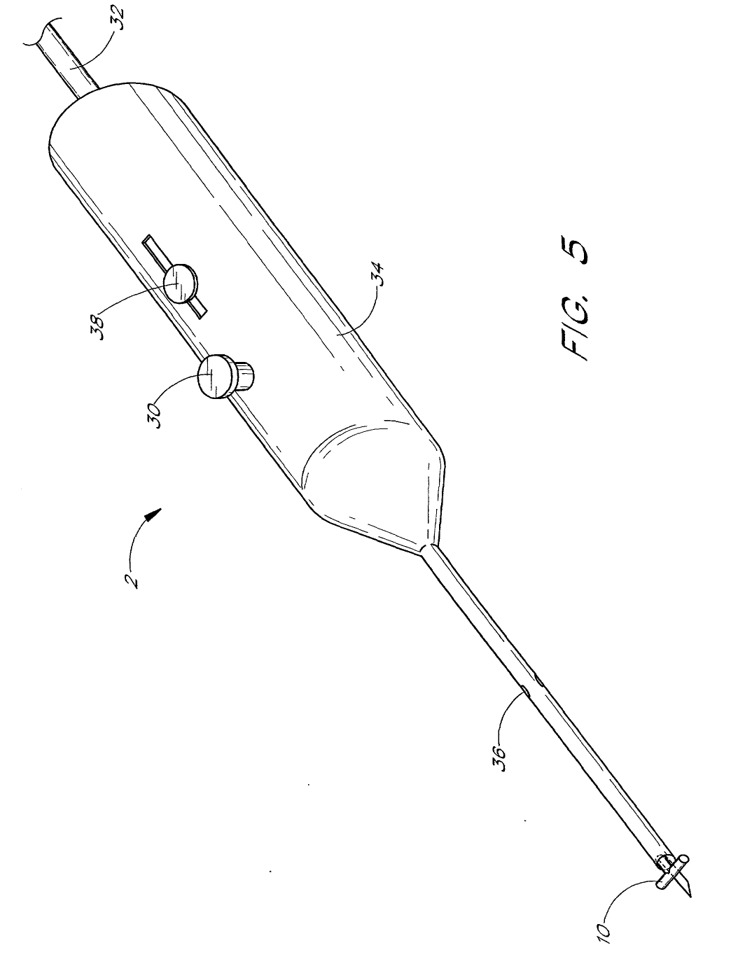

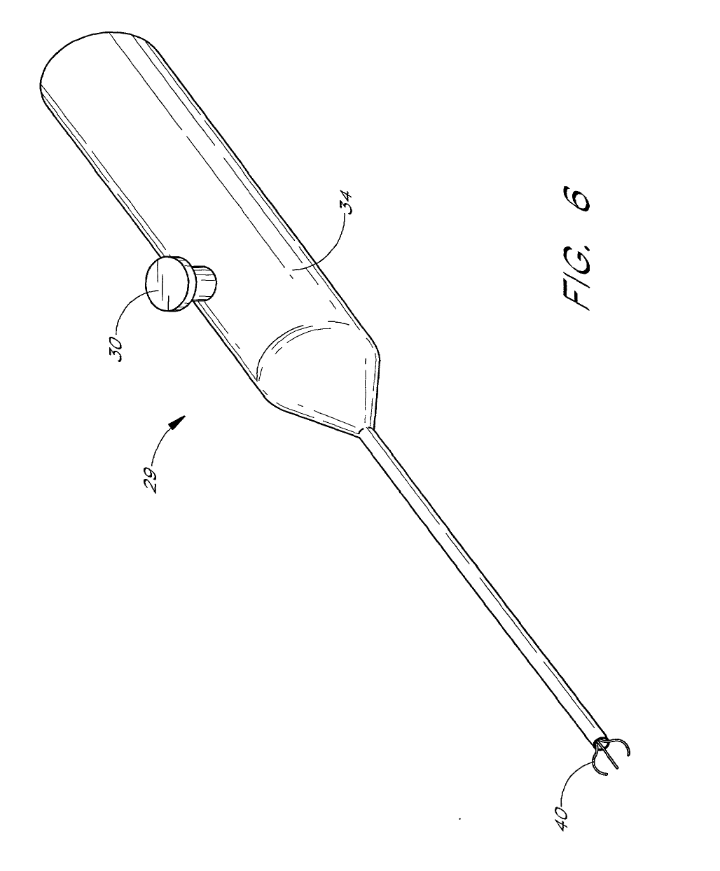

FIG. 5 illustrates one embodiment of the trabecular shunt applicator 2, holding the trabecular shunt 10 in place. Additionally, a trabecular meshwork blade 28 extends from the distal end of the applicator 2. In this embodiment, the blade 28 may be extended by spring action from the distal end of the applicator 2 when the operator pushes a button 30 or similarly actuates extension of the blade 28. The blade 28 can be retracted within the applicator 2 by means of a slide button 38, which the operator can move proximally to retract the blade 28. Alternatively, a plunger 32 may move the blade 28 forward and backward within the applicator 2. Also shown is the outer tube 34 of the applicator 2, as well as holes 36 in the applicator 2. These holes 36 may be used for aspiration or irrigation of the anterior chamber of the eye during the performance of trabecular meshwork surgery.

FIG. 6 illustrates one embodiment of a trabecular shunt retrieval device 29. To reacquire a shunt that is dropped in the anterior chamber requires the ability to grasp the shunt in a variety of orientations and from a variety of positions in the eye. Extending from the end of the retrieval device 29 is one or more claws 40 that can grasp the shunt 10. These claws may be extended from or retracted into the retrieval device 29. Actuation of these retractable claws 40 may be effected by an operator's push of a button 30 or engagement of any of a variety of other similar actuating devices that are known to those skilled in the art.

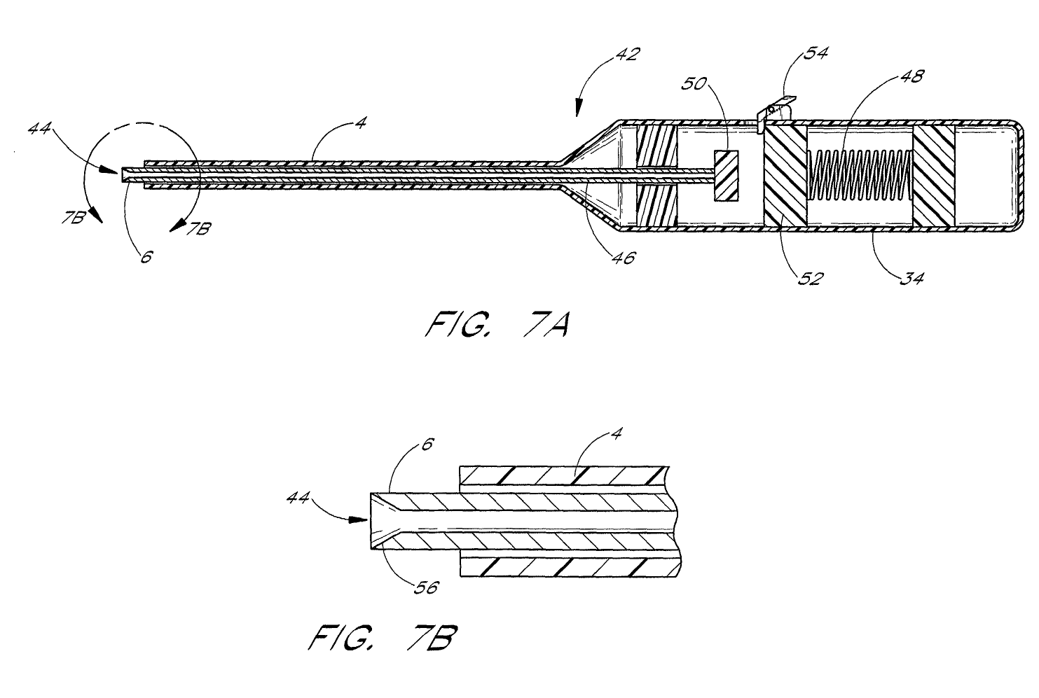

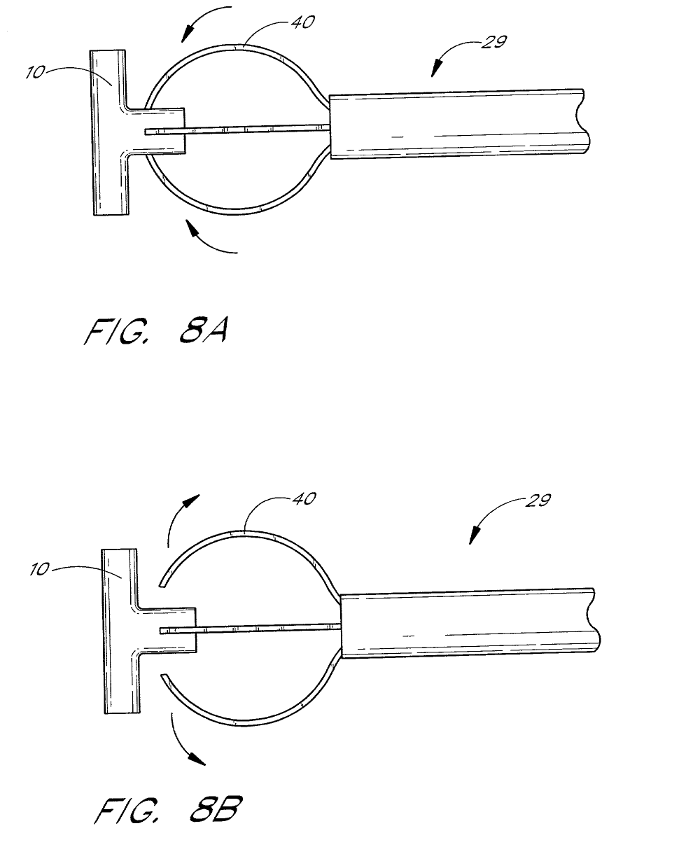

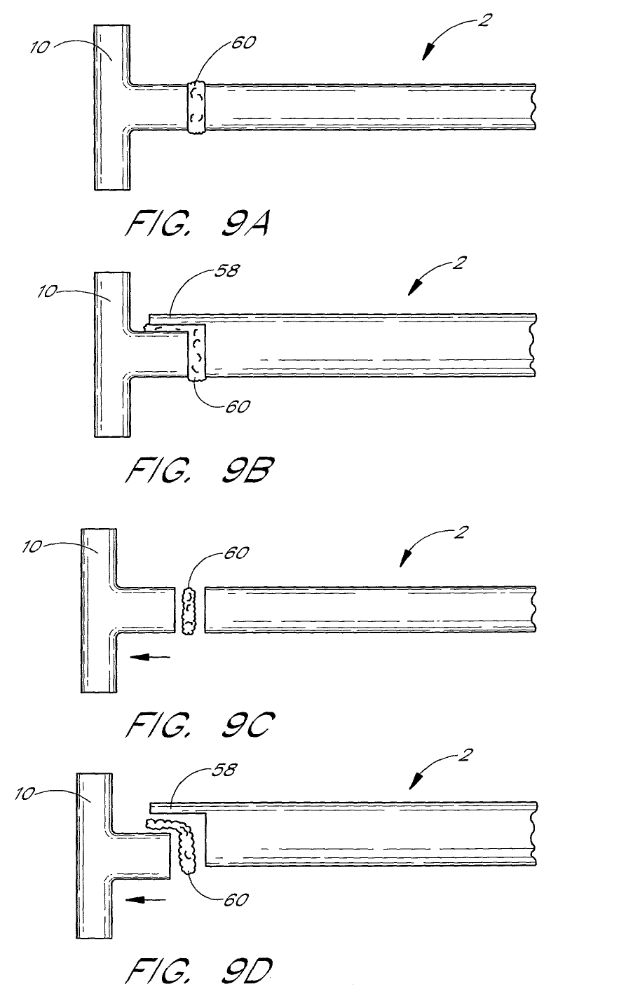

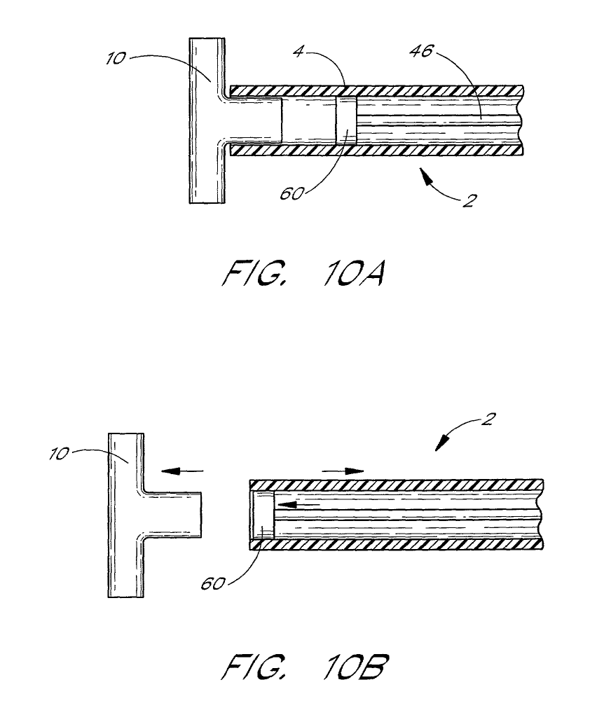

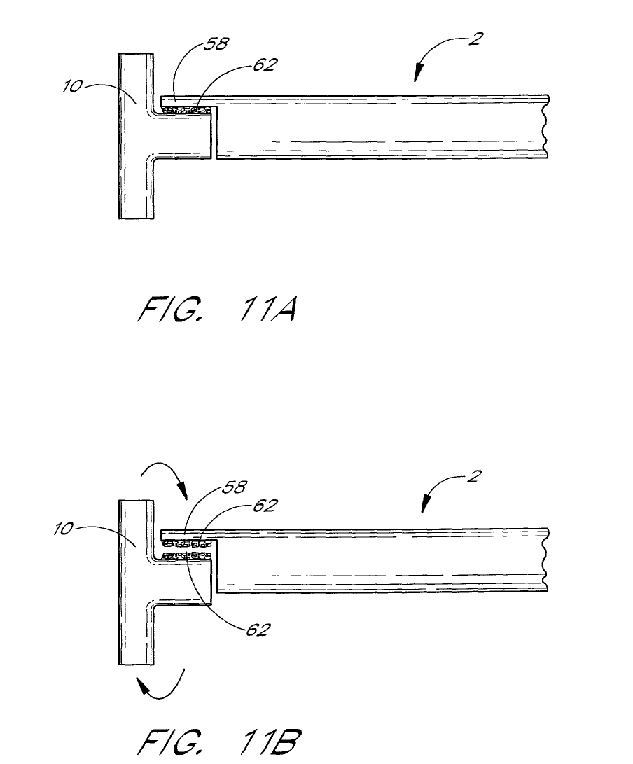

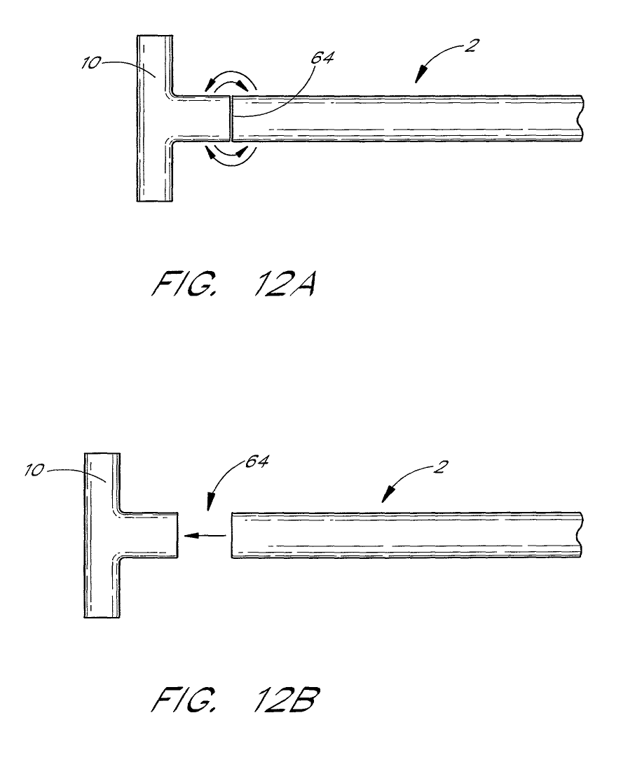

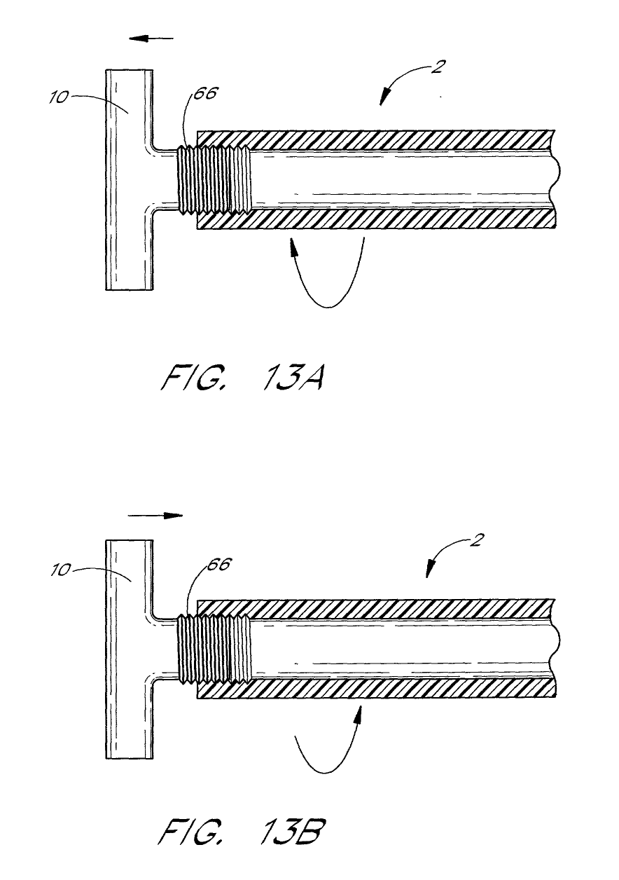

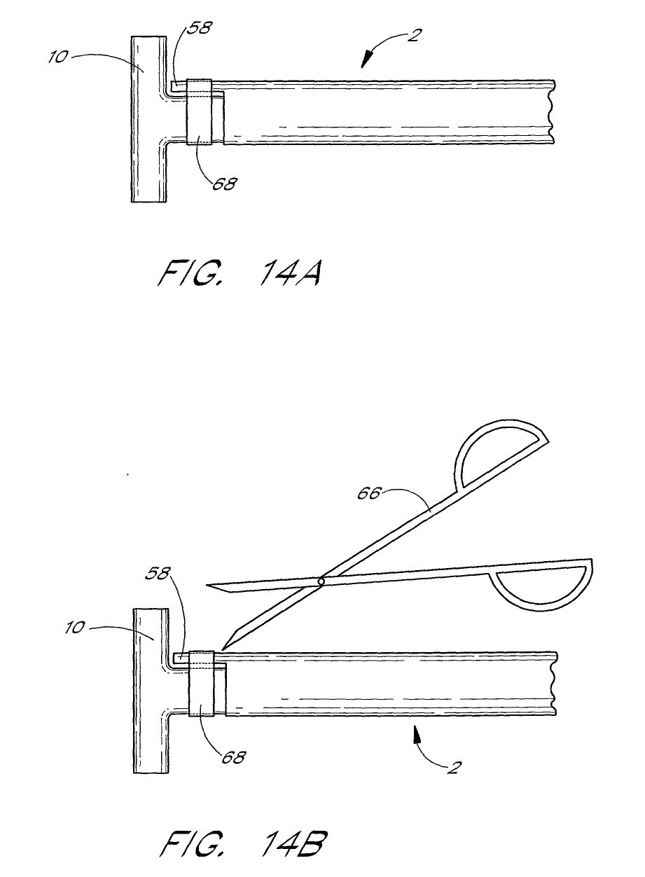

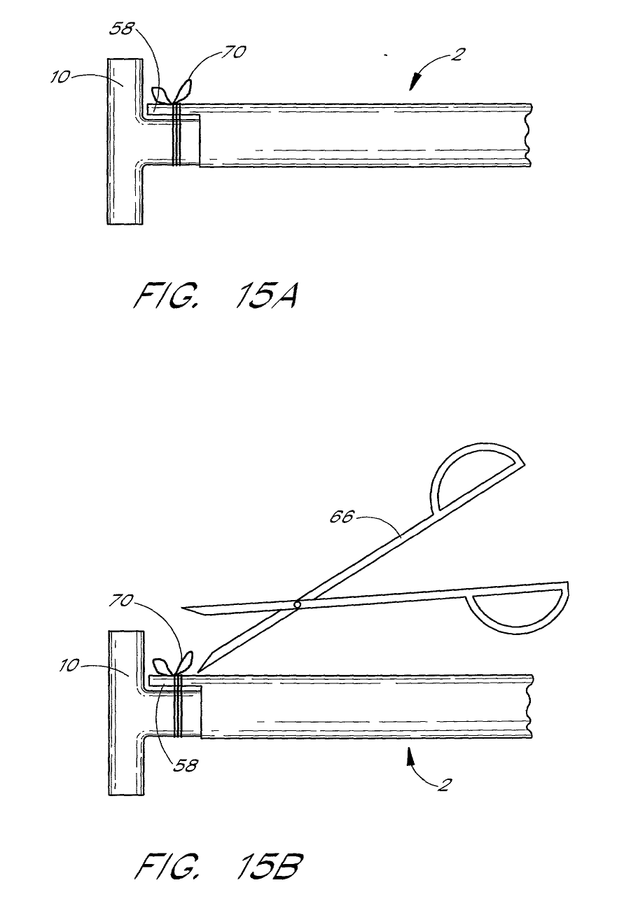

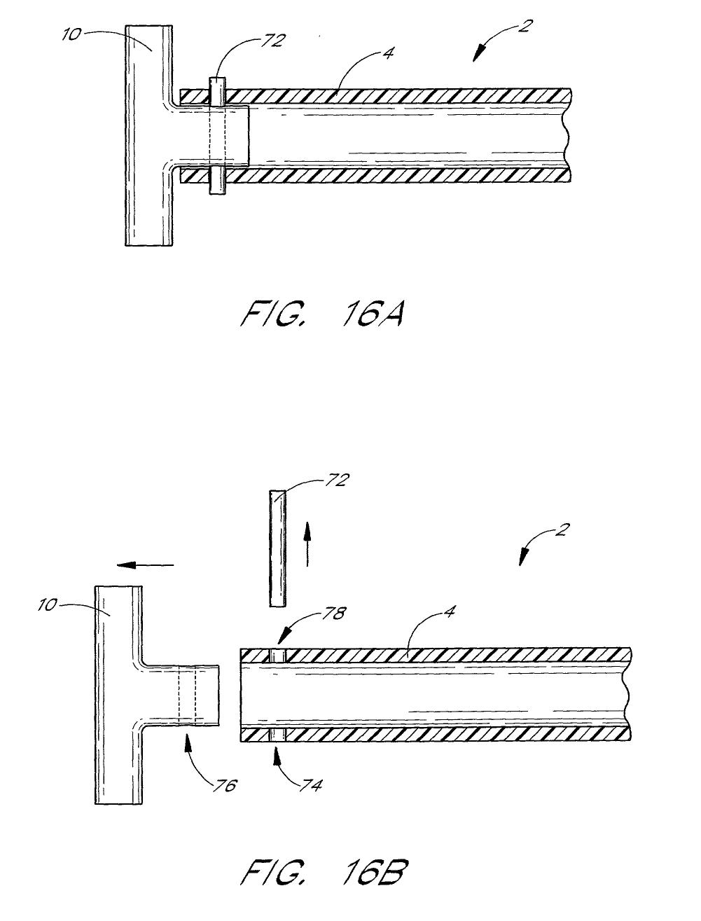

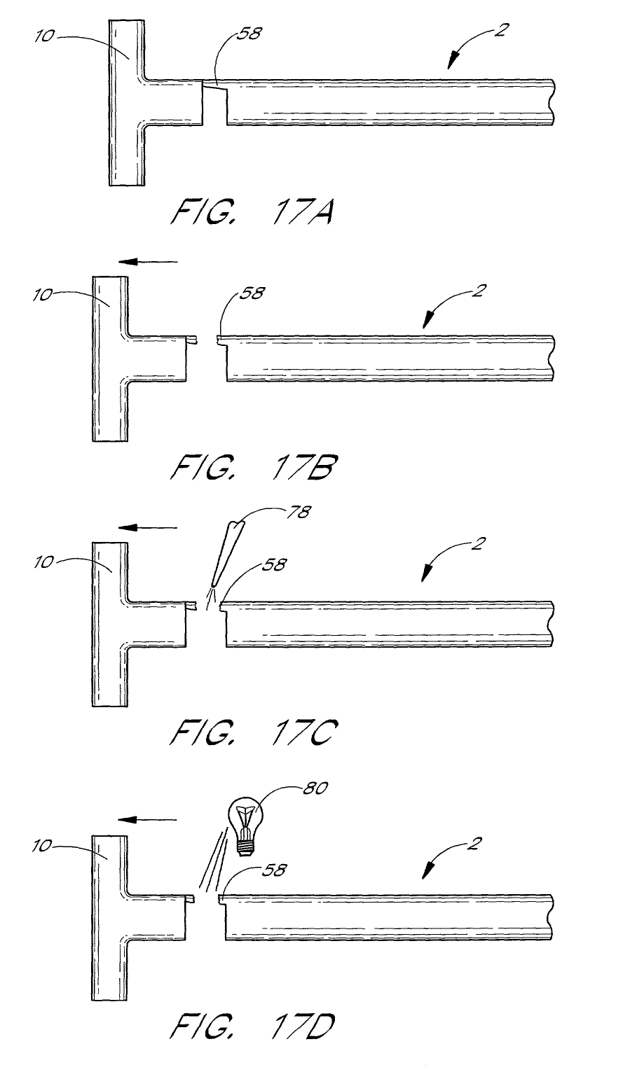

FIG. 7A shows one embodiment of a trabecular meshwork trephine, or punch 42. An inner tube 6 resides within an outer tube 4. The inner tube 6 is in communication with an inner plunger 46. The proximal end 50 of the inner plunger 46 is acted upon by a hammer 52 that is attached to a spring 48. The spring 48 may be recoiled or loaded, storing potential energy, and the hammer 52 is then held in place by an actuator 54 or other similar member in communication with the actuator 54. When the actuator 54 is acted upon by an operator, the spring 48 releases its potential energy, causing the hammer 52 to move forward, contacting the proximal end 50 of the inner plunger 46. This in turn causes the punch 44 to move forward, contacting the trabecular meshwork.

FIG. 7 view is a close-up, cross-sectional view of the punch 44. Again seen as the outer tube 4, the inner tube 6, and the punch 44 of the device. This trephine or punch may comprise a circular blade 56 or other similar configuration known to those skilled in the art for making a cut or punch hole in the trabecular meshwork of an eye.

FIGS. 8A and 8B demonstrate one embodiment of a trabecular shunt retrieval device 29. Again seen are the claws 40, which may hold the shunt 10 when the claw is partially retracted within the retrieval device 29. As illustrated in FIG. 8B, when the claws are extended from the retrieval device 29, a spring action within the claws 40 causes them to move away from the shunt 10 (curved arrows).

FIGS. 9A through 9D illustrate an adhesive mechanism for attaching and detaching the shunt 10 to the applicator 2.

In FIG. 9A, the adhesive 60 holds the shunt 10 to the applicator 2, in the sense that the adhesive 60 adheres to both the shunt 10, on one side, and the applicator 2 on another side. Once the adhesive is broken by various means, including traction, heat, and/or light, the shunt 10 moves away from the applicator 2, as illustrated in FIG. 9C.

FIG. 9B shows another embodiment of the adhesive mechanism. A protrusion 58 extending from the applicator 2 helps adherence of the applicator 2 to the shunt 10 by means of the adhesive 60. Once the adhesive bond between the shunt 10 and the applicator 2 is broken, as illustrated in FIG. 9D, the shunt may be left in place within the eye of the patient.

FIGS. 10A and 10B illustrates another embodiment of the applicator 2. In this embodiment, an inner plunger 46 is attached to a distal pusher 60. When the inner plunger 46 and distal pusher 60 move distally (left arrows) within outer tube 4, the distal pusher 60 comes in contact with the shunt 10 causing it to be pushed away from the outer tube 4. The shunt 10 may thence be left in the eye of the patient.

FIGS. 11A and 11B illustrate a hook-and-eye embodiment of a detachment mechanism for a trabecular shunt applicator 2. A hook-and-eye fastener 62 (such as Velcro™ or a miniaturized version of same) may be attached to a protrusion 58 on the applicator 2. When the applicator 2 is pulled away from the shunt 10 the two sides of the hook-and-eye fastener 62 come apart, leaving one side of the hook-and-eye fastener 62 attached to the shunt 10, in the other side of the hook-and-eye fastener 62 attached to the protrusion 58 of the applicator 2. In this fashion, the shunt 10 may be left within the eye of the patient, and the applicator 2 withdrawn from the eye.

FIGS. 12A and 12B illustrate a magnetic detachment mechanism for the trabecular shunt applicator 2. The applicator 2 and the shunt 10 are held together at a junction 64 by magnetic attraction (the magnetic fields shown stylistically by curved arrows), as illustrated in FIG. 12B. When the applicator 2 is moved away from the shunt 10, the magnetic “seal” between the applicator 2 and the shunt 10 at the junction 64 is broken, allowing the shunt 10 to be left behind in the patient's eye, when the applicator 2 is withdrawn from the eye.

FIGS. 13A and 13B illustrate another embodiment of the applicator 2. In this embodiment, the shunt 10 has screw threads 66 along one of its portions. These screw threads 66 fit into complementary threads in the applicator 2. When the surgeon desires to leave the shunt 10 in place within the eye of the patient, the surgeon may unscrew the applicator 2 from the shunt 10 by turning the applicator 2 in a counterclockwise or clockwise fashion (curved arrows).

FIGS. 14A and 14B illustrate another detachment mechanism for the trabecular shunt applicator 2. In this embodiment, an elastic band 68 holds the shunt 10 in place on the applicator 2 by wrapping around the shunt 10 and a protrusion 58 on the applicator 2. The surgeon may cut the elastic band 68, as illustrated in FIG. 14B, using a scissors 66 or similar cutting device as known to those skilled in the art. When the elastic band 68 is cut by the cutting instrument, such as the scissors 66, the elastic band breaks away from the protrusion 58 on the applicator 2 as well as the shunt 10. This allows the shunt 10 to be left in place in the eye and the applicator 2 to be withdrawn from the eye.

Another embodiment of a detachment mechanism is shown in FIGS. 15A and 15B. In this embodiment, a thread 70 or other tying device, such as a suture or string, is wrapped around the shunt 10 and the protrusion 58 on the applicator 2. The surgeon can cut the thread 60 using a scissors 66 or other similar cutting instrument, as illustrated in FIG. 15B. When the thread 70 is so cut, the applicator 2 may be withdrawn from the eye, leaving the shunt 10 in place within the eye.

FIGS. 16A and 16B demonstrate another detachment mechanism for the trabecular shunt 10 and the applicator 2. A pin 72 holds the shunt 10 in place within the outer tube 4 of the applicator 2. As illustrated in FIG. 16B, when the pin 72 is withdrawn from the outer tube 4 (upward arrow), the pin is removed from a hole 74 in the outer tube 4, as well as a shunt hole 76 in the shunt 10. This allows the applicator 2 to be moved away from the shunt 10, allowing the applicator 2 to be withdrawn from the eye while the shunt 10 remains in place within the eye.

FIGS. 17A through 17D illustrate various embodiments of detachment mechanisms for the trabecular shunt applicator 2. FIG. 17A illustrates an attachment to the shunt 10 of a protrusion 58 extending from the applicator 2. This protrusion 58 may connect to the shunt 10 via various means, such as by glue, welding or plastic fusion, or the molding or fabrication process. In FIG. 17B, the protrusion 58 has been broken, allowing the applicator 2 to move away from the shunt 10. The protrusion 58 may be broken in a variety of means, including, as shown in FIG. 17C, energy transfer from an energy source 78, such as a laser or thermal energy transferring device, as is well known to those skilled in the art. In FIG. 17D, a light source 80 can use ultraviolet light or other spectral frequencies to effect a chemical or electrochemical change in the protrusion 58 causing it to break. Once the light source 80 or other energy source 78 has broken the protrusion 58, the applicator 2 may be withdrawn from the eye, leaving the shunt 10 in place.

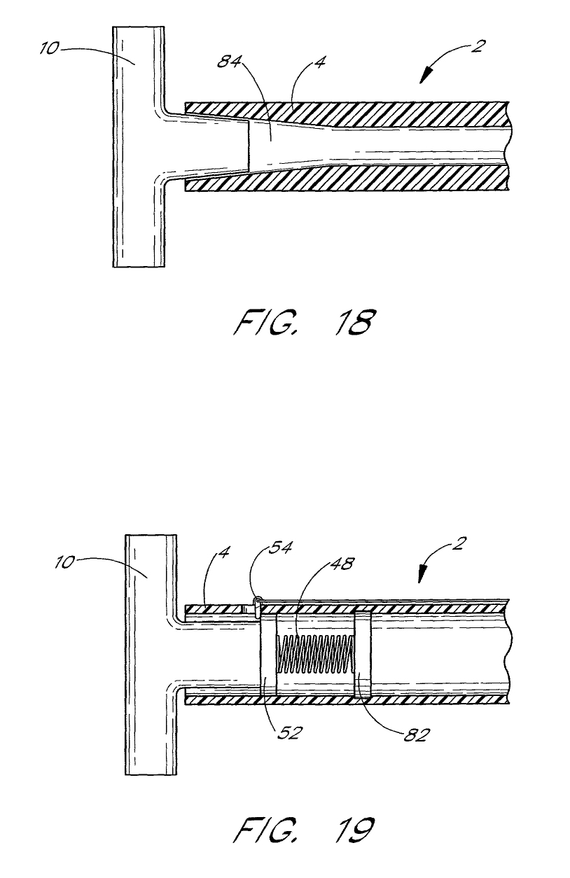

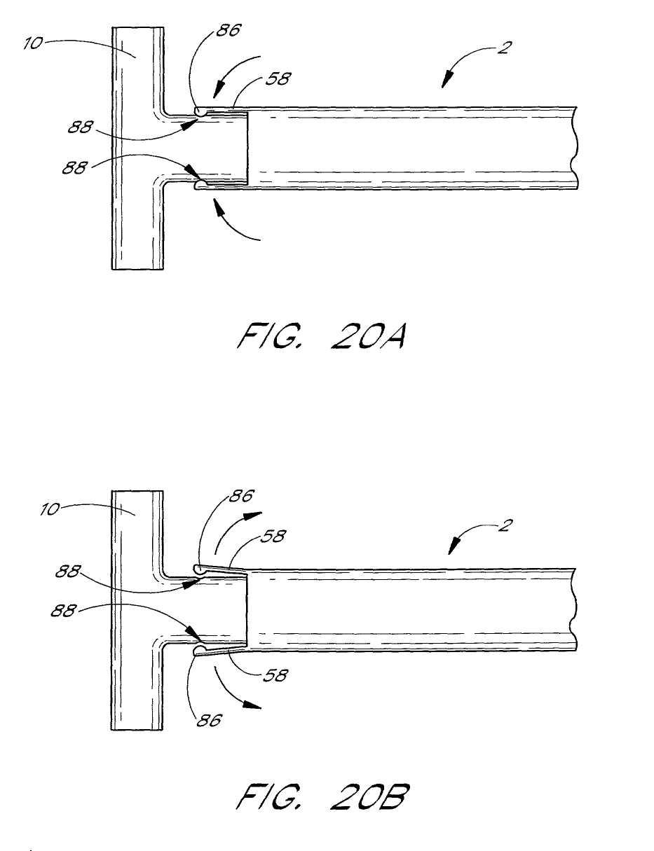

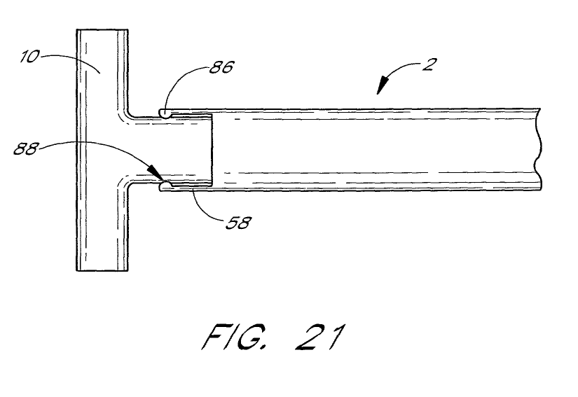

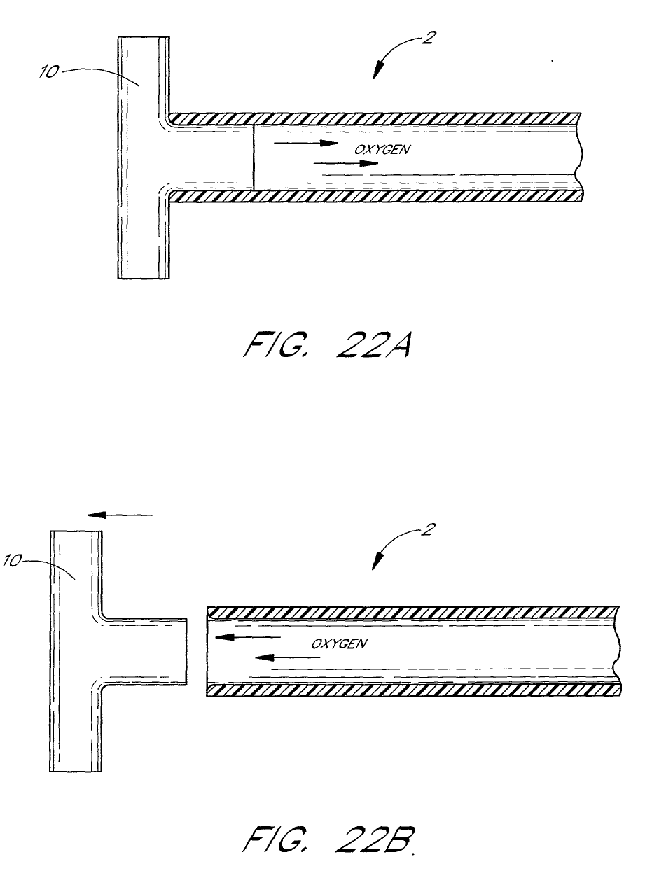

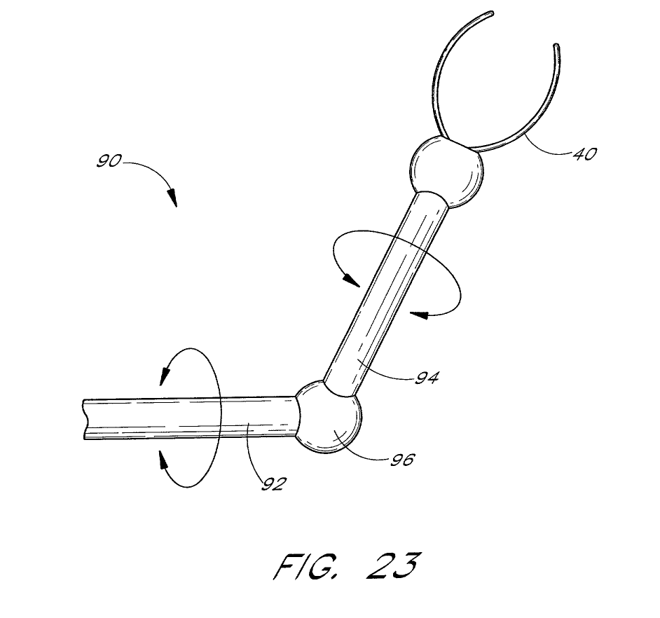

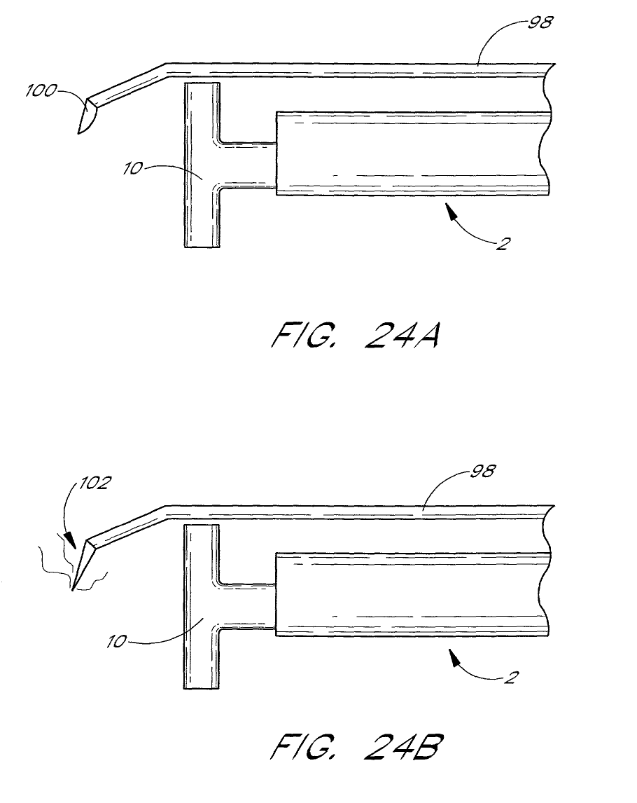

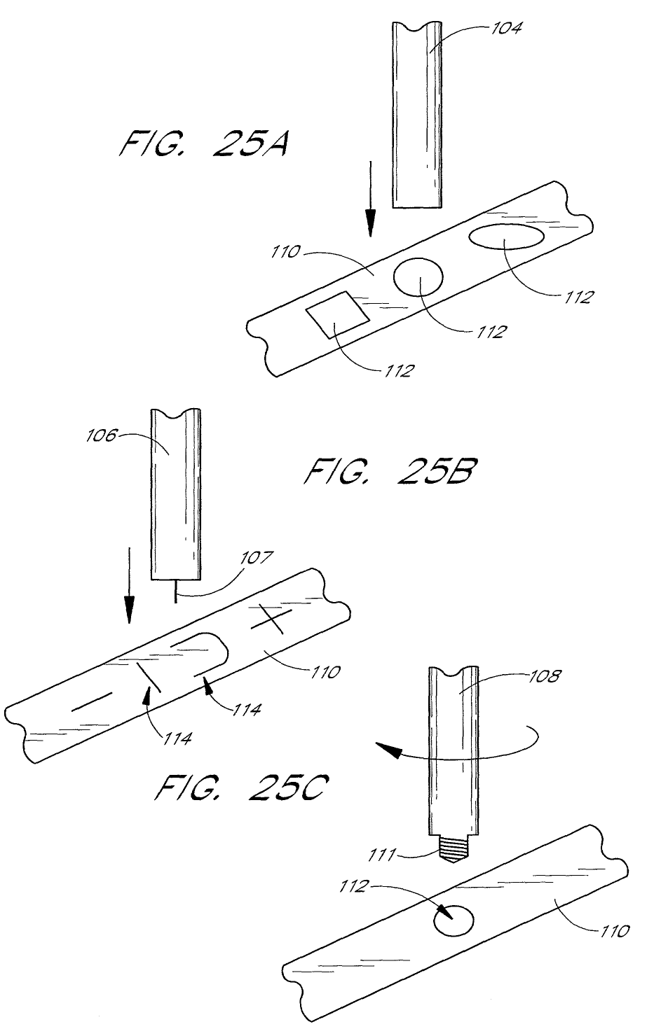

FIG. 18 illustrates a wedge-fit mechanism for the applicator 2. The outer tube 4 of the applicator 2 has a wedge configuration 84 within its lumen, and a similar wedge configuration in the inlet portion of the shunt 10 allows for a tight, “wedged,” fit for the shunt 10 within the applicator 2. Once the shunt 10 is in place within the eye, the applicator 2 may be moved away from the shunt 10, causing the shunt 10 to be dislodged from the outer wall 4 of the applicator 2 by virtue of the aforementioned wedge configuration 84 of the applicator 2 and shunt 10.

FIG. 19 illustrates a spring release mechanism for the applicator 2. In this embodiment, a hammer 52 is attached to a base 82 by a spring 48. When the spring 48 is loaded with energy, the hammer is then trapped in placed by an actuator 54 or other member in communication with the actuator 54. When the actuator 54 is actuated by an operator, the spring 48 is released, unloading its energy and driving the hammer 54 away from the base 82, toward the shunt 10. This drives the shunt 10 away from the outer wall 4 of the applicator 2, allowing it to be left in place within the eye. The applicator 2 may then be withdrawn from the eye.

FIGS. 20A and 20B illustrate another embodiment of a detachment mechanism for the trabecular shunt applicator 2. In this embodiment, one or more protrusions 58 extend from the applicator 2. One or more protuberances 86 extend from the protrusion 58. These protuberances 86 are preferably made of flexible plastic or rubber and can fit within one or more indentations 88 in the shunt 10. These protuberances 86 cause the shunt 10 to be held in place within the applicator 2 because the protuberances 86 fit within the indentations 88 in the shunt 10. When the surgeon pulls the applicator 2 away from the shunt 10 after the shunt 10 has been placed through the trabecular meshwork, the protuberances 86 are pulled out of the indentations 88 on the shunt, allowing the shunt 10 to break free of the applicator 2. Once the protuberances 86 slide out of the indentations 88 in the shunt 10, the applicator 2 may be withdrawn from the eye, while the shunt 10 remains in place within the eye.

FIG. 21 illustrates a similar embodiment of a detachment mechanism to that shown in FIGS. 20A and 20B. In this embodiment, the protrusions 58 are more rigid than that shown in FIGS. 20A and 20B, being made of semi-rigid plastic or metal, and the protrusions 58 extend from the applicator 2. One or more protuberances 86 extend from the protrusion 58. These protuberances 86 can fit within one or more indentations 88 in the shunt 10. These protuberances 86 cause the shunt 10 to be held in place within the applicator 2 because the protuberances 86 fit within the indentations 88 in the shunt 10. When the surgeon pulls the applicator 2 away from the shunt 10 after the shunt 10 has been placed through the trabecular meshwork, the protuberances 86 are pulled out of the indentations 88 on the shunt, allowing the shunt 10 to break free of the applicator 2. Once the protuberances 86 slide out of the indentations 88 in the shunt 10, the applicator 2 may be withdrawn from the eye, while the shunt 10 remains in place within the eye.

FIGS. 22A and 22B illustrate a suction detachment mechanism for the trabecular shunt applicator 2. In this embodiment, the shunt 10 is held in place within the applicator 2 by negative pressure, i.e., suction (right arrows). The suction may be provided by any suitable suction device as is well known to those skilled in the art. In FIG. 22B, the suction has been turned off and oxygen, air, or other suitable gas is allowed to flow into the applicator 2 (left arrows). This gas influx and consequent pressure change causes the shunt 10 to breakaway from the applicator 2, allowing the shunt 10 to break free of the applicator 2. This allows the shunt 10 to be left in place in the eye.

FIG. 23 illustrates one embodiment of an articulating applicator or retrieval device 90. In this embodiment, a proximal arm 92 is attached to a distal arm 94 at a joint 96. This joint 96 is movable such that an angle formed between the proximal arm 92 and the distal arm 94 can change. One or more claws 40 can extend from the distal arm 94, in the case of a shunt retrieval device. Similarly, this articulation mechanism may be used for the trabecular shunt applicator, and thus the articulating applicator or retrieval device 90 may be either an applicator for the trabecular shunt, a retrieval device, or both, in various embodiments.

FIGS. 24A and 24B illustrate embodiments of a control arm 98 which is attached to a mechanism for performing trabeculotomy. In FIG. 24A, a blade 100 extends from an end of the control arm 98. In some embodiments, the long axis of the control arm 98 runs parallel or semiparallel to the long axis of the applicator 2. The blade 100 may be used to make a trabeculotomy in preparation for placing the trabecular shunt 10 through the trabecular meshwork and into Schlemm's canal.

FIG. 24B shows a “hot tip” 102 at the end of the control arm 98. This hot tip may be a cautery, laser, or other energy transfer device for making a hole in the trabecular meshwork in preparation for placing the shunt 10 through the trabecular meshwork and into Schlemm's canal.

FIGS. 25A through 25C illustrate various embodiments of devices, such as trephines, that can punch holes in the trabecular meshwork. In FIG. 25A, a trabecular meshwork punch 104 is illustrated. This punch 104 can make holes 112 in the trabecular meshwork 110. These holes 112 can be of various configurations, depending on the shape of the distal blade of the trabecular meshwork punch 104.

In FIG. 25B, a blade 107 extends from the end of a trabecular meshwork cutter 106. This blade 107 can make various punch holes 114 in the trabecular meshwork 110, as illustrated.

FIG. 25C illustrates a trabecular meshwork drill 108. The drill 108 has a distal drill bit 111, which can make a drill hole 112 in the trabecular meshwork 110.

There are many alternatives for maintaining the anterior chamber during the installation of the trabecular shunt 10, including the irrigating, irrigating side port, over-fill, viscoelastic, and air bubble.

Additionally, there are many alternatives for creating a trabecular meshwork incision. Of these, the punch, stab, drill, and shunt alternatives are likely to create surgeon-independent, repeatable incisions. The ideal size of the shunt 10 is based on the size of the Schlemm's canal that it is inserted into and on the size of the hole in the trabecular meshwork. A surgeon-independent incision would help ensure that the shunt fits well despite who is performing the surgery. Of these surgeon-independent alternatives, the punch and drill remove material that will leave room for the outlet portion of the shunt without having to create overlaps or folds in the trabecular meshwork tissue. The drill alternative creates debris and is therefore perhaps less desirable than the punch. The sharp shunt alternative is enticing, since it removes the need to cross the anterior chamber twice; however, the sharp tip may potentially do damage to the inside of Schlemm's canal or may lead to inappropriate placement of the shunt.

There are multiple alternatives for creating a corneal incision, including the micro-knife.

Due to the anatomy of trabecular meshwork being in a curved ring configuration inside the eye, and in view of the ab interno approach within the confined space of the anterior chamber, the tip section of the trephine for creating an opening within the trabecular meshwork may be angled. An angled-tip trephine may, in some circumstances, more easily enable creating an opening in the trabecular meshwork suitable for inserting a the glaucoma shunt more easily into Schlemm's canal.

While inserting a glaucoma shunt through the trabecular meshwork into Schlemm's canal in an ab interno procedure, it is desirable to cause minimal injury to Schlemm's canal. Therefore, one consideration for creating an opening using a trephine is to limit its penetrating distance in Schlemm's canal. The trabecular meshwork is generally about 200 to 400 microns. Some embodiments provide a depth-limited microtrephine adapted for cutting through at least a major portion of the trabecular meshwork, while not injuring the back (outer) surface of Schlemm's canal.

To further simplify the operation of creating an opening in the trabecular meshwork, one aspect provides an automated microtrephine, which, by a touch of a button at the handpiece, permits a predetermined cutting force and/or cutting distance, thereby eliminating much of an operator's chance for error in creating an opening.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A delivery apparatus for placing a trabecular shunt through a trabecular meshwork of an eye, the shunt having an inlet section and an outlet section, the delivery apparatus comprising:
a handpiece having a distal end and a proximal end;
an elongate tip connected to the distal end of said handpiece, said elongate tip having a distal portion and being configured to be placed through a corneal incision and into an anterior chamber of said eye;
a holder attached to the distal portion of the elongate tip, the holder configured to hold and release said inlet section of the trabecular shunt; and
an actuator on the handpiece that actuates the holder to release the inlet section of the trabecular shunt from the holder.

2. The delivery apparatus of claim 1, wherein said holder comprises a clamp.

3. The delivery apparatus of claim 1, further comprising a spring within the handpiece that is configured to be loaded when said shunt is being held by said holder, said spring being at least partially unloaded upon actuating said actuator, allowing for release of said shunt from said holder.

4. The delivery apparatus of claim 2, wherein the clamp comprises a plurality of claws configured to exert a clamping force onto the inlet section of said shunt.

5. The delivery apparatus of claim 1, wherein said holder comprises a plurality of flanges.

6. The delivery apparatus of claim 1, wherein the distal portion of the elongate tip is made of a flexible material.

7. The delivery apparatus of claim 6, wherein the distal portion of the elongate tip is made of a flexible wire.

8. The delivery apparatus of claim 6, wherein the distal portion has a deflection range of about 45 degrees from a long axis of the handpiece.

9. The delivery apparatus of claim 1, further comprising an irrigation port in the elongate tip.

10. A method of placing a trabecular shunt through a trabecular meshwork of an eye, the shunt having an inlet section and an outlet section, comprising:
advancing a delivery apparatus holding the trabecular shunt through an anterior chamber of said eye and into the trabecular meshwork, placing part of the shunt through the trabecular meshwork and into a Schlemm's canal of said eye; and
releasing the shunt from said delivery apparatus.

11. The method of claim 10, wherein the delivery apparatus comprises:
a handpiece having a distal end and a proximal end;
an elongate tip connected to the distal end of said handpiece, said elongate tip having a distal portion and being configured to be placed through a corneal incision and into an anterior chamber of said eye;
a holder attached to the distal portion of the elongate tip, the holder configured to hold and release said inlet section of the trabecular shunt; and
an actuator on the handpiece that actuates the holder to release the inlet section of the trabecular shunt from the holder.
